(19) United States
(12) Patent Application Publication
(10) Pub. No.: US 2023/0215516 A1
(43) Pub. Date: Jul. 6, 2023

(54) JOINT MULTI-NANOPORE SEQUENCING FOR RELIABLE DATA RETRIEVAL IN NUCLEIC ACID STORAGE

(71) Applicant: QUANTUM CORPORATION, San Jose, CA (US)

(72) Inventors: Suayb S. Arslan, Cambridge, MA (US); Turguy Goker, Oceanside, CA (US); Don Doerner, San Jose, CA (US)

(21) Appl. No.: 18/092,654

(22) Filed: Jan. 3, 2023

Related U.S. Application Data
(60) Provisional application No. 63/296,805, filed on Jan. 5, 2022.

(51) Int. Cl. G16B 40/10; G01N 33/487; C12Q 1/6869

(52) U.S. Cl. CPC G16B 40/10; G01N 33/48721; C12Q 1/6869

(57) ABSTRACT

A nucleic acid storage system (100) that uses nanopore sequencing to read data values chemically embedded in oligonucleotides includes a membrane (102), a voltage source (108), and a nucleic acid strand (110). The membrane (102) has a plurality of nanopores (104) that are stacked upon one another in a multi-nanopore arrangement. The voltage source (108) is configured to direct voltage across the plurality of nanopores (104). The nucleic acid strand (110) including the oligonucleotides is threaded through each of the plurality of nanopores (104) within the membrane (102). A separate base signal (118) is generated from the nucleic acid strand (110) being threaded through each of the plurality of nanopores (104), and Recursive Neural Networks can be used to estimate a signal shape for each oligonucleotide. Recurrent Convolutional Neural Networks and noise predictive data detection algorithms can be used based on the estimated signal shapes to sequence the oligonucleotides.

### RELATED APPLICATION

This application claims priority on U.S. Provisional Application Ser. No. 63/296,805 filed on Jan. 5, 2022 and entitled “JOINT MULTI-NANOPORE SEQUENCING FOR RELIABLE DATA RETRIEVAL IN NUCLEIC ACID STORAGE”. As far as permitted, the contents of U.S. Provisional Application Ser. No. 63/296,805 are incorporated in their entirety herein by reference.

### BACKGROUND

DNA (deoxyribonucleic acid), or RNA (ribonucleic acid) digital data storage is the process of encoding and decoding binary data to and from synthesized strands of DNA (or RNA). According to a recent study, just four grams of DNA could store all of the world's digital data for a year. The capacity to store ten times more data, a thousand-fold storage density, and a 108-fold reduction in power consumption when storing the same amount of data are all qualities that DNA offers. Before DNA (or RNA) can be utilized as a future data storage technology/platform, a number of challenges must be solved, including exorbitant costs, painfully slow writing and reading processes, and sensitivity to mutations or errors. Stated in another manner, while DNA (or RNA) as a storage medium has enormous potential because of its high storage density, its practical use is currently severely limited because of its high cost, very slow read and write times, and sensitivity to error.

Data writes and reads are named synthesis and sequencing, respectively, in the DNA data storage terminology. In reality, the end-to-end process entails converting digital data to DNA sequences, manipulating biomolecules physically, storing them, and then retrieving the data by sequencing the DNA. DNA sequencing is the process of determining the nucleic acid sequence, i.e. the order of nucleotides in DNA. It includes any method or technology that is used to determine the order of the four bases: adenine (“A”), guanine (“G”), cytosine (“C”), and thymine (“T”). There are different types of sequencing methods, grouped as the first, second, and third generation. For instance, Illumina sequencing is based on a sequencing method using reversible dye-terminators technology, and engineered polymerases, known as the second generation. Although the accuracy of such is relatively high (with error rates on the order of 0.01 and lower), the read sequence lengths are only on the order of hundreds. Plus, the process can be slow, thereby limiting the data read and access rates.

The third generation is typically based on nanopore sequencing, which is a more cost-effective solution. Moreover, it is quite inexpensive to prepare a sample, requiring minimal chemistries or enzyme-dependent amplification. Furthermore, a nanopore sensor eliminates the need for nucleotides and polymerases or ligases during readout. Despite the advantages, there are many challenges ahead for the proliferation of nanopore sequencing technology and to become part of the DNA drives of the future.

Nanopore sequencing is a method for DNA data storage and is used to read data values chemically embedded in oligonucleotides. In particular, using nanopore sequencing, a single molecule of DNA can be sequenced without the need for PCR amplification or chemical labeling of the sample. In nanopore sequencing, a biological or solid-state membrane, where the nanopore is found, is surrounded by an electrolyte solution. In such a technique, a strand of DNA molecules passes through a specially designed pore (either biological or solid-state) and a voltage is applied across the pore which ends up creating an electrical field across pore ends. This voltage (the field itself) creates an ionic current to pass through the pore (movement of charges due to the field). Depending on the type of the molecule passing through the pore, different current blockade levels and translocation speeds can be measured and recorded through placing electrodes near the membrane. Based on various factors such as pore geometry, size and chemical composition, the change in the magnitude of the ionic current blockade and the duration of the translocation (so called dwell time) will vary over time.

As noted, there are two types of nanopore sequencing: Biological and Solid-state. Biological nanopore sequencing makes use of porins, which are transmembrane proteins embedded in lipid membranes that form size-dependent porous surfaces with nanometer-scale “holes” scattered across the membranes. Some best-known biological examples include Alpha hemolysin, which uses a nanopore from bacteria that causes lysis of red blood cells, and Mycobacterium smegmatis porin A (MspA), which has been identified as a potential improvement over Alpha hemolysin due to a more favorable structure.

Unlike biological nanopore sequencing, solid-state nanopore sequencing does not include proteins in its structure. Solid-state nanopore technology, on the other hand, employs a variety of metal or metal alloy substrates with nanometer-sized holes that allow DNA to flow through in a controlled process. Some most notable approaches are based on either current blockade or tunneling, which entails measurement of electron tunneling through bases as single-stranded DNA translocates through the nanopore, or fluorescence, which entails converting each base into a characteristic representation of multiple nucleotides which bind to a fluorescent probe strand-forming double-stranded DNA.

Both technologies have their own pros and cons, with biological nanopore sequencing having an advantage in (i) low translocation velocity (defined as the speed at which a sample passes through a unit's pore slowly enough to be measured) and (ii) dimensional reproducibility (defined as the likelihood of a unit's pore to be made the proper size); and solid-state nanopore sequencing having an advantage in (iii) stress tolerance (defined as the sensitivity of a unit to internal environmental conditions), (iv) longevity (defined as the length of time that a unit is expected to remain functioning), and (v) ease of fabrication (defined as the ability to produce a unit, usually with regard to mass-production). Furthermore, there are hybrid nanopore sequencing technologies that combine biological and solid-state approaches at the same time.

A main objective of the detection process is to be able to differentiate different nucleotides based on the uniquely generated current blockade levels. In the blockade or current-tunneling method, each level of ionic current maps to a k-mer (a k-base long base sequence such as ATCGC is one 5-mer example sequence). In bioinformatics, k-mers are substrings of length k contained within a biological sequence. Primarily used within the context of computational genomics and sequence analysis, in which k-mers are composed of nucleotides (such as adenine (A), guanine (G), cytosine (C) and thymine (T), for DNA), k-mers are capitalized upon to assemble DNA sequences, improve heterologous gene expression, identify species in metagenomic samples, and create attenuated vaccines. Usually, the term k-mer refers to all of a sequence's subsequences of length k, such that the sequence AGAT would have four monomers (A, G, A, and T), three 2-mers (AG, GA, AT), two 3-mers (AGA and GAT) and one 4-mer (AGAT). More generally, a sequence of length L will have L-k+1 k-mers and n^{k} total possible k-mers, where n is the number of possible monomers (such as four in the case of DNA). These k-mers share a prefix with the suffixes of a previous k-mer for a given nanopore (k is determined based on the nanopore's depth). Since k-mers have short-term (long-term) dependencies, researchers tend to model it as a language (and different k-mers as words, etc.) and hence use the most-fitting artificial intelligence (AI) approach namely Recursive Neural Networks (RNNs) to do the base-calling (base sequencing or base detection).

Assuming that the electrical fields inside pores are capable of generating unique blockade levels based on different type of bases, the classical approach to base detection (so called base-calling, where molecule types (a.k.a. bases) (A, G, C, T) are detected) has been Recursive Neural Networks (RNNs), whose main objective is to learn about the time-series data that has distinct ionic current blockade levels and translocation speeds for different oligonucleotides. However, RNN is typically applied to the current level signals and the expected output is the nucleotide type, hence abstracting the entire DNA read channel end-to-end. Though such an approach may work well in practice, it makes it impossible to reason about how the detection works in the data retrieval process, let alone the design of detection and signal processing steps. In addition, besides its complexity, current RNN-based detection error rates (without any error correction techniques applied, etc.) levels around 0.10-0.15 error rate values, which makes it impractical to use DNA as a reliable storage medium where the end goal is to achieve overall user error rates better than 1 error in 10¹⁹ user bits, which is a typical user bit error rate number for enterprise or LTO class tape magnetic storage. Moreover, evidence exists that the noise/interference characterization for DNA read channel shows major colored-ness that seems to be solved by the RNN also in the detection process. There are many time-variant disturbances associated with the channel, but all are supposed to be learned and solved by the RNN itself, which strictly ties the detection with the availability of data for training and resources for computation/processing. Finally, the lack of knowledge about the overall detection process essentially inhibits users from manufacturing genuine pores to help with the recording as well as the reading (base detection) process.

One of the fundamental issues with the classical approach is that it is unclear whether the present data error-rate is fundamental to the nanopore (indifferentiable output for different bases or base sequences) or due to the limitations of present base-calling algorithms. In fact, in the limiting case when k is large, the number of possible k-base combinations will be so large that differentiation based on ionic current level would be impossible. That is why recent research is increasingly focusing on extremely small pores and single-base detection at a given time. However common, the key to this problem is to come up with a realistic channel model, which is currently impossible to do due to the use of neural networks. Modeling such channel/signals is pretty complicated due to the following four important reasons/observations:

1) The output at any given time depends on k>1 bases (k-mers), and so there is inter-symbol interference which may be quite non-linear.

2) There may be collisions in the output particularly for large k: two different pore contents may lead to similar/the same current readouts that might be too confusing to be intelligible/separable (low spatial resolution).

3) On top of the signals, there is also filtered/colored noise (unlikely to be Gaussian).

4) The amount of time that each k-mer spends in the pore can vary, and sometimes may never occur at all, leading to synchronization errors, deletions or insertions in the output (random translocation speeds—low temporal resolution).

This type of channel/signal characterization would be incredibly helpful in benchmarking base-calling algorithms and determining what is and is not possible. More significantly, state-of-the-art nanopore sequencers may well be sub-optimal, implying that their chemical development process, architecture, and component placements are not optimized to aid base-calling (data detection) algorithms in the best manner possible.

It is further appreciated that any such system that employs nanopore sequencing is likely to experience complexity of implementation. Any commercial device with nanopore sequencing capability will come with multiple physical nanopores laid out in a two-dimensional grid/membrane that would define independent channels for parallel processing of DNA molecules. For instance, one previous device has 512 independent channels allowing 512 different DNA molecules to be sequenced all at the same time. Associated with each one of the channels is a neural network that processes and detects nucleotides, and which needs to be trained at specific periodic intervals to update its parameters for best base-calling performance. In fact, to do a consensus read (multiple reads of the same data), these networks have to run multiple times (or a separately bigger network designed for consensus) for the same sequence. Finally, in the future generations of such sequencers it is likely to have 10000×10000 nanopores with millions of processing units to be able to increase the data access rates for DNA drives/storage devices. However, having 100 million different neural networks (and training each one of them) inside the device makes it practically infeasible for even testing. At some point, running such a huge number of networks even for testing/classification purposes may be burdensome from an implementation point of view. Future neuromorphic hardware may be applicable here, however its commercialization cost will rule it out as a possible candidate using today's technology.

Thus, it is desired to provide techniques to make nanopore sequencing a viable option for future practical use cases by reducing complexity, reducing cost, improving read and write times, and reducing sensitivity to error.

### SUMMARY

The present invention is directed to a nucleic acid digital data storage system that uses nanopore sequencing to read data values chemically embedded in oligonucleotides. In various embodiments, the nucleic acid digital data storage system includes a membrane, a voltage source, and a nucleic acid strand. The membrane has a plurality of nanopores that are stacked upon one another in a multi-nanopore arrangement. The voltage source is configured to direct voltage across the plurality of nanopores. The nucleic acid strand including the oligonucleotides is threaded through each of the plurality of nanopores within the membrane.

In some embodiments, the nanopores are surrounded by an electrolyte solution within the membrane.

Although the invention is generally described in detail herein in relation to DNA digital data storage, it is appreciated that substantially the same systems and methods would be equally applicable utilizing RNA in lieu of DNA or other configurations where digital 0's and 1's are encoded using any combination of four bases that make up the genetic code, which for DNA are adenine (A), guanine (G), cytosine (C) and thymine (T). Therefore, it is not intended that the scope of the present disclosure be limited in such manner.

In particular, in certain embodiments, the nucleic acid strand is a DNA strand, and the oligonucleotides include one or more of adenine, guanine, cytosine, and thymine.

In other embodiments, the nucleic acid strand is an RNA strand.

In some embodiments, the voltage from the voltage source is applied across each of the plurality of nanopores independently of one another to create an electrical field across pore ends of each of the plurality of nanopores. In certain embodiments, the electrical field creates an ionic current to pass through each of the plurality of nanopores.

In certain embodiments, the membrane is usable to capture multiple waveforms for a base sequence when the oligonucleotides are threaded through the plurality of nanopores. In some embodiments, the oligonucleotides being threaded through each of the plurality of nanopores generates a corresponding ionic current.

In some embodiments, a separate base signal is generated from the nucleic acid strand being threaded through each of the plurality of nanopores. In certain embodiments, Recursive Neural Networks can be used to estimate a signal shape for each oligonucleotide. In some embodiments, Recurrent Convolutional Neural Networks and noise predictive data detection algorithms can be used based on the estimated signal shapes to sequence the oligonucleotides.

In certain embodiments, each of the base signals is modified by one or more of a post-processing (PP) system, a joint symbol detection system, and an Error Correction Coding (ECC) decoding system. In other embodiments, each of the base signals is modified by each of the post-processing system, the joint symbol detection system, and the ECC decoding system. In one embodiment, each of the base signals is modified by the post-processing system, prior to being subjected to the joint symbol detection system and the ECC decoding system.

In some embodiments, the post-processing system utilizes one or more of an adaptive filter, a shifter, a data padding system, an aperiodic sampling system, and a whitening filter to modify the base signals.

In certain embodiments, the joint symbol detection system includes one or more of a branch metric calculator and a trellis.

In some embodiments, the ECC decoding system includes one or more of an insertion-deletion (indel) decoder and a secondary error correction decoder.

In certain embodiments, the plurality of nanopores includes a first nanopore, a second nanopore and a third nanopore that are stacked one on top of another from top to bottom in the multi-nanopore arrangement. In some embodiments, the membrane further includes a first cavity that is defined between the first nanopore and the second nanopore, and a second cavity that is defined between the second nanopore and the third nanopore.

In some embodiments, each of the plurality of nanopores is a different size from each of the other nanopores.

In certain embodiments, each of the plurality of nanopores has a different translocation speed than each of the other nanopores. In one embodiment, the first nanopore has the highest translocation speed, the second nanopore has the next highest translocation speed, and the third nanopore has the slowest translocation speed.

In some embodiments, the first cavity has a first size, and the second cavity has a second size that is different than the first size.

In certain embodiments, the nucleic acid strand is a double-helix DNA strand.

In some embodiments, the membrane is a biological membrane. In other embodiments, the membrane is a solid-state membrane. In still other embodiments, the membrane is a hybrid of a biological membrane and a solid-state membrane.

The present invention is further directed toward a method for using nanopore sequencing to read data values chemically embedded in oligonucleotides, the method including the steps of stacking a plurality of nanopores upon one another in a multi-nanopore arrangement within a membrane; directing voltage across the plurality of nanopores with a voltage source; and threading a nucleic acid strand including the oligonucleotides through each of the plurality of nanopores within the membrane.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic illustration of an embodiment of a nucleic acid digital data storage system having features of the present invention;

FIG. 2 is a simplified schematic illustration of a portion of the nucleic acid digital data storage system illustrated in FIG. 1, including an embodiment of a membrane, a voltage source and a DNA strand;

FIG. 3 is a simplified schematic illustration of an embodiment of a post-processing system that can be incorporated into the nucleic acid digital data storage system illustrated in FIG. 1;

FIG. 4 is a simplified schematic illustration of an embodiment of a joint symbol detection system that can be incorporated into the nucleic acid digital data storage system illustrated in FIG. 1;

FIG. 5 is a simplified schematic illustration of an embodiment of an Error Correction Coding (ECC) decoding system that can be incorporated into the nucleic acid digital data storage system illustrated in FIG. 1;

FIG. 6 is a representative graphical illustration of a base signal estimation for nanopore sequencers that may be seen using the nucleic acid digital data storage system illustrated in FIG. 1; and

FIG. 7 is a simplified schematic cross-sectional view illustration of nanopores usable within the nucleic acid digital data storage system illustrated in FIG. 1 shown on a two-dimensional planar surface.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described in the context of a nucleic acid digital data storage system (also sometimes referred to as a “data storage system” or simply a “storage system”) that utilizes joint multi-nanopore sequencing for reliable data retrieval. More particularly, in various embodiments, the data storage system is configured to use multiple-pore manufacturing in the same membrane to capture multiple waveforms for the same base sequence. In other words, the same oligonucleotides pass through multiple physically collocated pores (stacked on top of each other) with potentially different translocation speeds, and each generates a corresponding ionic current. As referred to herein, it is appreciated that a nanopore is a pore of nanometer size. Thus, the terms “nanopore” and “pore” are sometimes used interchangeably herein.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar reference indicators will be used throughout the drawings and the following detailed description to refer to the same or like parts.

In the interest of clarity, not all of the routine features of the implementations described herein are shown and described. It will, of course, be appreciated that in the development of any such actual implementations, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

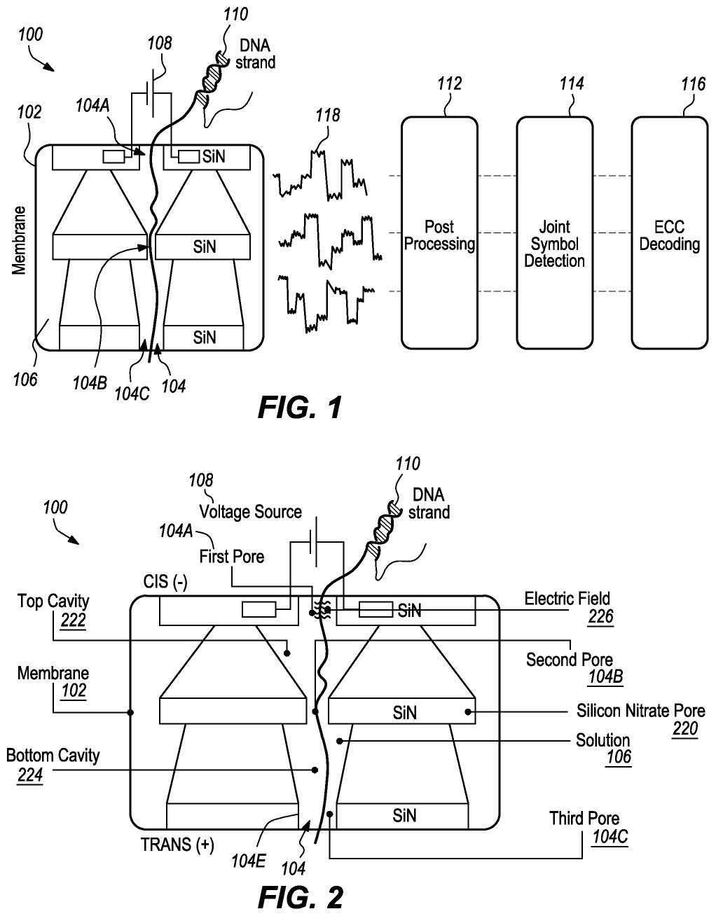

In various implementations of the present invention, the data storage system is configured to use multiple nanopores (with each individual nanopore being either biological (protein-based), solid-state, or a hybrid thereof) with different aperture sizes and potentially chemical content (protein, graphene, silicon nitrate, etc.), usable in nanopore sequencing for reliable data retrieval. An example structure of the multi-pore cross-section, as well as the subsequent system components, is shown in FIG. 1. More specifically, FIG. 1 is a simplified schematic illustration of an embodiment of a nucleic acid digital data storage system 100 (also referred to as a “data storage system” or simply as a “storage system”) including a membrane 102 (either a biological membrane, a solid-state membrane, or a hybrid thereof) having a plurality of nanopores 104 (or “pores”) that are stacked upon one another in a multi-nanopore arrangement, which are surrounded by an electrolyte solution 106; a voltage source 108; and a nucleic acid strand, such as a DNA strand 110 in this non-exclusive embodiment, that is threaded through the membrane 102, such as through the nanopores 104 positioned within the membrane 102; and further including a post-processing system 112, a joint symbol detection system 114 (also referred to herein as a “detection system”), and an Error Correction Coding (ECC) decoding system 116 (also referred to herein as a “decoding system”). With such design, as described in greater detail herein, the same membrane 102 can be used to capture multiple waveforms for the same base sequence, with the same oligonucleotides passing through multiple physically collocated nanopores 104 with potentially different translocation speeds, and each generating a corresponding ionic current. Additionally, or in the alternative, the data storage system 100 can include more components or fewer components than what is illustrated in FIG. 1.

DNA-based data storage systems encode digital information (typically in a series of 0's and 1's) using combinations of the four nucleotides (adenine (A), guanine (G), cytosine (C) and thymine (T), more commonly known as “bases”) of which DNA is composed. There is considerable flexibility in that encoding. For example, each base may represent two bits, or individual (or short sequences of) bits may be represented by short, predetermined sequences of bases. It is recognized that the systems and methods described in detail herein are applicable in all of these cases.

Although the invention is generally described in detail in relation to DNA digital data storage, it is appreciated that substantially the same systems and methods would be equally applicable utilizing RNA in lieu of DNA. Therefore, it is not intended that the scope of the present disclosure be limited in such manner.

It is appreciated that the membrane 102 can include any suitable number of nanopores 104 that are stacked one upon another. For example, in the embodiment illustrated in FIG. 1, the membrane 102 includes three nanopores 104, such as a first (upper) nanopore 104A, a second (middle) nanopore 104B, and a third (lower) nanopore 104C, which are stacked upon one another in a multi-nanopore arrangement. Alternatively, the membrane 102 can include greater than three nanopores 104 or only two nanopores 104 in accordance with the teachings of the present invention.

In different implementations, the nanopores 104 may, for example, be created by a pore-forming protein or as a hole in synthetic materials such as silicon or graphene. More particularly, as noted, the nanopores 104 can be biological, solid-state, or a hybrid thereof. In one such implementation, the nanopores 104 are created as holes in silicon nitrate (SiN) structures and/or materials.

As further illustrated in FIG. 1, base signals 118 that are generated from the DNA strand 110 being threaded through the nanopores 104 are also shown, as the base signals 118 are then moved through, subjected to, processed, detected, decoded and/or modified by the post-processing system 112, the detection system 114, and the decoding system 116. More particularly, in summary, a multi-nanopore storage system 100 as described leads to a sequence of read-out base signals 118, and the three modules, such as the post-processing system 112, the detection system 114, and the decoding system 116 in this particular embodiment, process these raw base signals 118 to be able to decide on the final DNA molecule.

The post-processing undertaken within the post-processing system 112 can take different shapes depending on the signal quality, signal synchronization, signal amplitude, signal phase among other properties of the signal captured. There may also be coupling between the nanopore currents due to the physical proximity which will be compensated in the joint symbol detection system 114 after post-processing is done. Finally, the data is decoded using generated redundancy (ECC) within the decoding system 116.

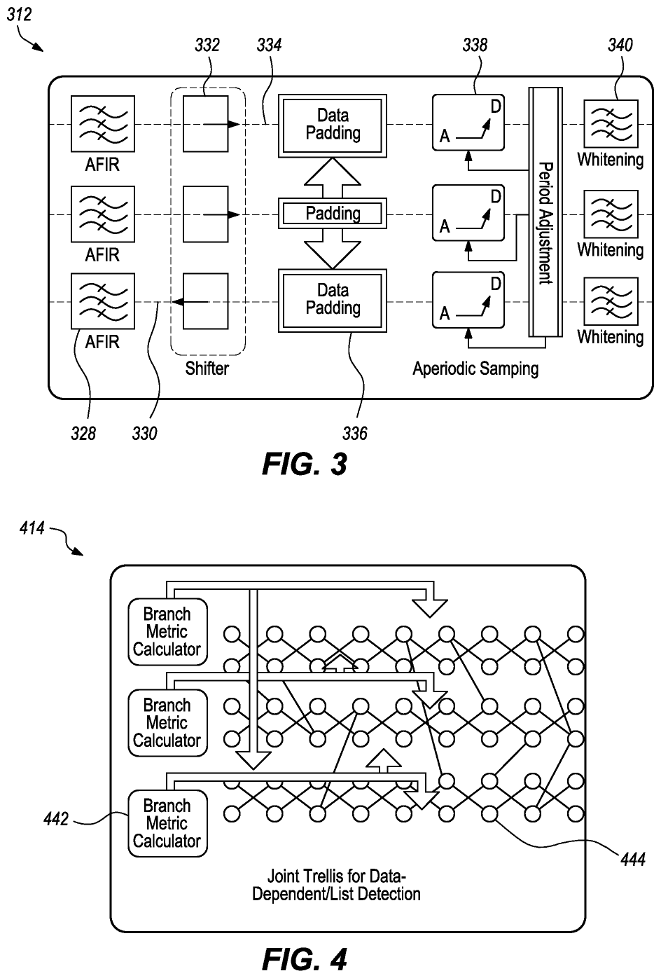

Each of the major components of the embodiment of the storage system 100 of FIG. 1, including the membrane 102 and the various components included therein, the post-processing system 112, the detection system 114 and the decoding system 116, are shown in greater detail in FIGS. 2-5 herein below. Initially, details of an embodiment of the membrane 102 and the various components utilized therein is illustrated in FIG. 2. Subsequently, details of embodiments of the post-processing system 112, the joint symbol detection system 114, and the ECC decoding system 116 of the data storage system 100 are illustrated in FIGS. 3, 4 and 5, respectively.

FIG. 2 is a simplified schematic illustration of a portion of the nucleic acid digital data storage system 100 illustrated in FIG. 1, including an embodiment of the membrane 102, the voltage source 108 and the DNA strand 110.

As noted above, the membrane 102 can be provided in the form of either a biological membrane, a solid-state membrane, or a hybrid thereof. In one non-exclusive embodiment, the membrane 102 can include silicon nitrate structures 220 that form the plurality of nanopores 104.

In various embodiments, the membrane 102 includes the plurality of nanopores 104 (or “pores”) that are stacked upon one another in a multi-nanopore arrangement. The nanopores 104 are further surrounded by the electrolyte solution 106. For simplicity of illustration, in the embodiment specifically illustrated in FIG. 2, the membrane 102 includes three nanopores 104 that are stacked one upon another in the multi-nanopore arrangement. However, it is appreciated that the membrane 102 can include any suitable number of nanopores 104, which may be greater than three nanopores 104 or only two nanopores 104. As further shown in FIG. 2, the size and shape of each of the plurality of nanopores 104 can be varied. More specifically, in this non-exclusive embodiment, the first (upper) nanopore 104A, the second (middle) nanopore 104B, and the third (lower) nanopore 104C are shown as each having a slightly different size and shape.

The areas within the membrane 102 between the nanopores 104 can also be referred to as cavities. For example, as shown in FIG. 2, a first (top) cavity 222 is defined between the first nanopore 104A and the second nanopore 104B, and between the uppermost and middle silicon nitrate structures 220; and a second (bottom) cavity 224 is defined between the second nanopore 104B and the third nanopore 104C, and between the middle and lowermost silicon nitrate structures 220. As shown, the cavities 222, 224 may be different sizes from one another. With such design, the present invention provides the ability to control the translocation time of DNA molecules through the use of multiple nanopores 104 which may be interleaved with different sized cavities 222, 224.

It is appreciated that the nanopores 104 are again illustrated in FIG. 2 as being surrounded by the electrolyte solution 106.

When one or more nanopores 104 are present in an electrically insulating membrane 102, a detection principle is based on monitoring the ionic current passing through the nanopores 104 as a voltage is applied across the membrane 102. When the nanopores 104 are of molecular dimensions, passage of molecules (such as DNA) cause interruptions of the “open” current level, leading to a “translocation event” signal.

As illustrated, in a nanopore sequencing technique, which is used to read data values chemically embedded in oligonucleotides, the DNA strand 110 passes through the plurality of nanopores 104 and voltage from the voltage source 108 is applied across the nanopores 104 which ends up creating an electrical field 226 across pore ends 204E (one such electrical field 226 is identified in FIG. 2). This voltage (the electrical field 226 itself) creates an ionic current to pass through the nanopores 104 (movement of charges due to the electrical field 226). The effect of applying a bias voltage across the membrane 102 thereby inducing the electrical field 226 that drives charged particles, in this case the ions, into motion, is known as electrophoresis. For high enough concentrations, the electrolyte solution 106 is well distributed and all the voltage drop concentrates near and inside the nanopores 104. This means charged particles in the electrolyte solution 104 only feel a force from the electrical field 226 when they are near the pore region. This region is often referred to as the capture region.

Inside the capture region, ions have a directed motion that can be recorded as a steady ionic current by placing electrodes near the membrane 102. More particularly, as noted above, depending on the type of the molecule passing through the nanopores 104, different current blockade levels and translocation speeds can be measured and recorded through placing electrodes near the membrane 102. This molecule also has a net charge that feels a force from the electrical field 226 when it is found in the capture region. The molecule approaches this capture region aided by Brownian motion and any attraction it might have to the surface of the membrane 102. Once inside the nanopore 104, the molecule translocates through via a combination of electro-phoretic, electro-osmotic and sometimes thermo-phoretic forces. Inside the nanopore 104, the molecule occupies a volume that partially restricts the flow of ions, observed as an ionic current drop. Different molecules can then be sensed and potentially identified based on this modulation in ionic current. For example, based on various factors such as nanopore 104 geometry, size and chemical composition, the change in the magnitude of the ionic current blockade and the duration of the translocation (so called dwell time) may vary over time.

The voltage source 108 can be any suitable type of voltage source that is configured to provide the desired voltage across the nanopores 104 which ends up creating the electrical field 226 across the pore ends 204E, and which creates the ionic current to pass through the nanopores 104.

As illustrated in FIG. 2, in various embodiments, the DNA strand 110 can be a double-helix DNA strand that is fed into the nanopores 104. An enzymatic reaction dispatches the strands and one of them passes through the three different nanopores 104A-104C, which can have different sizes and chemical content and distinct cavity 222, 224 volumes/rooms. The translocation speed also varies due to natural manufacturing differences between the nanopores 104, cavity 222, 224 sizes and the type of motor mechanism (such as a protein) used to move the DNA strand 110 or some other mechanism. The first nanopore 104A assumes the fastest speed, whereas as one moves down the membrane 102, the average translocation speed of the nanopores 104 decreases. A voltage from the voltage source 108 is applied across each nanopore 104 independently. This voltage leads to induced ionic current blockade through the nanopores 104 which are measured and recorded.

In the real-time streaming, these base signals 118 (illustrated in FIG. 1) are post-processed within the post-processing system 112 (illustrated in FIG. 1) after the ionic current is measured and recorded.

FIG. 3 is a simplified schematic illustration of an embodiment of the post-processing system 312 that can be incorporated into the nucleic acid digital data storage system 100 illustrated in FIG. 1. The post-processing undertaken within the post-processing system 312 can take different shapes depending on the signal quality, signal synchronization, signal amplitude, signal phase among other properties of the base signals 118 (illustrated in FIG. 1) that have been captured in the manner as described above.

As illustrated, in certain embodiments, the raw base signals 118 first go through a bank of adaptive filters 328 (such as Adaptive Finite-Impulse Response filters (AFIRs) or other suitable types of filters) in parallel, whose coefficients are subject to optimization/learning, to generate a plurality of filtered signals 330. Next, due to physical separation between the nanopores 104 (illustrated in FIG. 1) and varying translocation, shifting operation within one or more shifters 332 is applied to each one of the filtered signals 330 depending on their location in the stacked architecture to generate a plurality of shifted signals 334. The shifter 332 does signal shifts (either to the right or to the left) to generate the shifted signals 334. The closer the filtered signal 330 is to the center, the less the amount of shift becomes.

Following this stage, data is padded as necessary onto the shifted signals 334 with a data padding system 336 due to the shifting operation. Data padding is used to place zeros for frame completion in some embodiments. Subsequently, the waveform is sampled within an aperiodic sampling system 338 at a period that can change over time (adjusted based on the translocation and physical distances or geometries). In other words, sampling within the sampling system 338 creates samples from the signals subject to non-uniform sampling periods. Finally, a whitening filter 340 is used to change the statistical properties of the colored noise. This whitening filter 340 is typically designed to be a finite-impulse response filter also, but can alternatively include another suitable type of filter such as an infinite impulse response (IIR) filter. The whitening filter 340 operates on the discrete samples and helps the subsequent detection process minimally affected by the colored nature of the noise. Such a sequence of post processing tools prepares the signal samples for the subsequent detection process.

FIG. 4 is a simplified schematic illustration of an embodiment of a joint symbol detection system 414 that can be incorporated into the nucleic acid digital data storage system 100 illustrated in FIG. 1. The detection process uses branch metric calculations for each signal. Therefore, there is a branch metric calculator 442 before the data passes through a trellis 444 that is configured for use in data-dependent list detection. To embed data-dependency, at the expense of complexity, for multiple potential data sequences, different branch metrics can be calculated. The trellis 444 is constructed and branch metrics are used to calculate a proximity metric. The trellis 444 can alternatively be constructed jointly and hence jumping from one trellis 444 to another might be possible as shown in FIG. 4. Based on the accumulated branch metrics on the trellis 444, a most likely path is found through a standard backtracking. If more memory is used to keep track of multiple most likelihood paths in each step of the trellis 444, then a group of most likely S sequences can be generated for each nanopore 104 (illustrated in FIG. 1) by following the valid paths on the joint trellis 444. This list approach can help improve the detection accuracy. Data dependency can be inserted into the branch metric calculator 442 module for each possible data sequence, and a different branch metric can be calculated and used for different branches at different times.

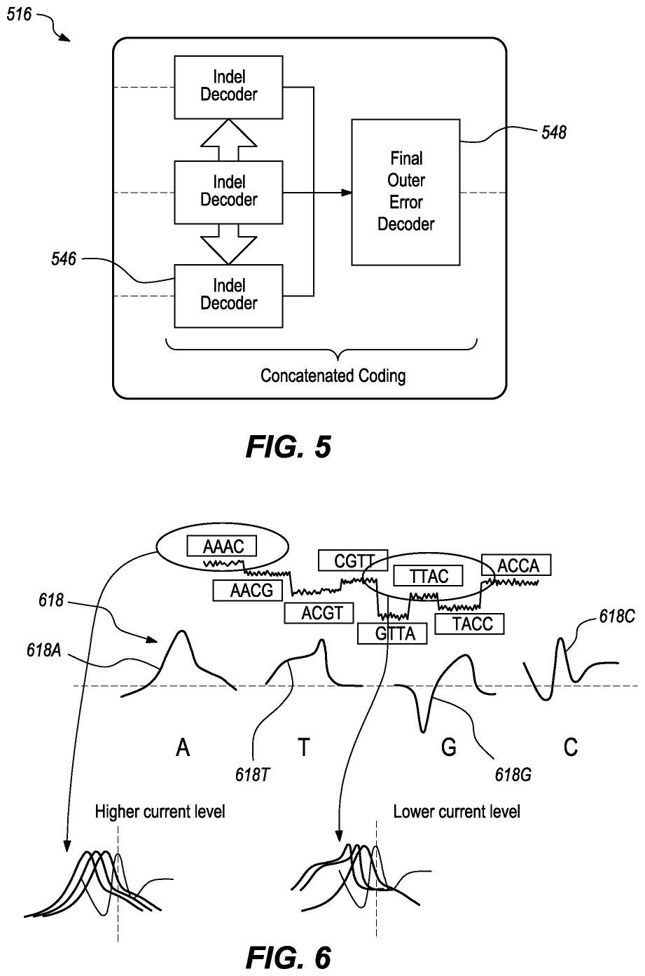

FIG. 5 is a simplified schematic illustration of an embodiment of an ECC decoding system 516 that can be incorporated into the nucleic acid digital data storage system 100 illustrated in FIG. 1. Despite the fact that the storage system 100 is configured to optimize the alignment between different nanopore read-outs, the nanopores 104 (illustrated in FIG. 1) themselves may miss or insert new nucleotides due to varying translocation speed or imperfections inside the cavities 222, 224 (illustrated in FIG. 2) or nanopores 104 (illustrated in FIG. 1). Thus, symbols may be inserted, deleted (indel for short), or substituted. Thus, an individual indel decoding is applied to each detection output. Due to the correlation between distinct detection outputs, these indel decoders 546 work collaboratively and pass information among themselves to increase the accuracy of the symbol/data correction. The remaining substitution errors are resolved by a concatenated error and/or erasure decoding algorithm. This final decoder 548 combines the results of the indel decoder 546 outputs, merges them and minimizes the number of errors before running the secondary error correction decoder algorithm. The main purpose of the final decoder 548 is to pull the error rates to 10\hat{ }-19 or below at the worst case. The code rates for each coding stage in a concatenated setting are determined based on the nominal uncoded error rate of the storage system 100. This would be a function of nanopores used, detection algorithm parameters, preprocessing tools employed and environmental conditions, among other effects.

It is appreciated that the joint symbol detection system 414 and the ECC decoding system 516 that can be incorporated as part of the nucleic acid digital data storage system 100 can include features, components and details somewhat similar to what was illustrated in the bit error detection and correction system of U.S. patent application Ser. No. 13/719,777 filed on Dec. 19, 2012 that utilizes a combination of a List-Viterbi (or “List-NPMLD”) detection algorithm, and error detection code decoders for reducing the number of error events at the output of the Viterbi (or “NPMLD”). As far as permitted, the contents of U.S. patent application Ser. No. 13/719,777 are incorporated in their entirety herein by reference.

In summary, after the base signals 118 are collected in the manner illustrated and described, post-processing is applied to the collected current waveforms. Following the post-processing, a joint detector architecture follows to generate the final base-calling output before implementation of the Error Correction Coding (ECC) decoding stage. To correctly operate, it is necessary to have a decent signal model and a PP+detector combination that should be implemented carefully based on the operating conditions and the resulting data. Various methods of post-processing and detection methods are provided as a list of claims in the following. Each of these claims can either alone or jointly be implemented to address the problems previously mentioned herein.

In a first claim, in order to enhance understanding of the channel, reduce complexity, and decouple different stages of the data detection process, it is proposed to use Artificial Neural Networks/Recursive Neural Networks (ANN/RNN) to estimate isolated impulse responses of the nanopore to four different bases, namely A, G, C and T. In this characterization, each ionic current level is a result of multiple signals shifted right/left and superimposed on each other. An example scenario is illustrated and described in greater detail herein above. With this treatment, simple threshold-detector approaches can be designed based on the signal shapes as well as severity of the inter-symbol-interference. Alternative detection methods can also be proposed, of which some are detailed in other claims.

In a second claim, in an embodiment of the present invention, it is assumed that the response of a given nanopore to a nucleotide is a combination of two channel responses h₁(t) and h₂(t). To model the varying translocation, time shifts of these two signals are assumed to form the current blockade signal,

I(t)=Σᵢaᵢhᵢ(t−iT)+bᵢh₂(t−iS)+η(t) (Equation 1)

where aᵢ∈{+1, −1} and bᵢ∈{+1, −1}. Also, T and S are the periods for these responses and η(t) is the noise component of the observed current signal I(t). There are four combinations of aᵢbᵢ which are used to encode nucleotides A, G, C and T. In this formulation, h₁(t), h₂(t), T and S are estimated based on the given recorded signals so that given the DNA sequence I(t) most mimics the training data. There may be multiple AI-based approaches to the estimation process. In one embodiment, neural networks can be used, whereas in the other, linear or non-linear regression techniques can alternatively be used.

FIG. 6 is a representative graphical illustration of a base signal estimation for nanopore sequencers that may be seen using the nucleic acid digital data storage system illustrated in FIG. 1. As shown in FIG. 6, each of the nucleotides, or bases, A, G, C and T, has a unique estimated base signal shape that is found through use of the process of nanopore sequencing. More particularly, as shown, the adenine (A) nucleotide has a first estimated base signal shape 618A, the thymine (T) nucleotide has a second estimated base signal shape 618T that is different than the first estimated base signal shape 618A, the guanine (G) nucleotide has a third estimated base signal shape 618G that is different than the first estimated base signal shape 618A and the second estimated base signal shape 618T, and the cytosine (C) nucleotide has a fourth estimated base signal shape 618C that is different than the first estimated base signal shape 618A, the second estimated base signal shape 618T and the third estimated base signal shape 618G.

With the base signals 118 (one example of which is shown in FIG. 6) generated through threading the DNA strand 110 (illustrated in FIG. 1) through the nanopores 104 (illustrated in FIG. 1) within the membrane 102 (illustrated in FIG. 1), a base sequence is generated that relates to the current level which includes a concatenation of four individual signal shapes. Examples are illustrated in FIG. 6 for sequence “AAAC” and sequence “TTAC”.

In certain embodiments, Recursive Neural Networks (RNNs) are used to estimate the signal shapes for each base nucleotide rather than using a base detection process directly. Based on the estimated signal shapes, the data storage system is configured to use Recurrent Convolutional Neural Networks (R-CNNs) and conventional detection algorithms based on estimated signal shapes such as noise predictive maximum likelihood detection (NPMLD) to sequence the nucleotides in a spatially coordinated way. In this manner, improved detection accuracy performance is ensured, while giving a brand-new methodology to the detection process within the context of explainable AI and low-complexity information decoding.

Assuming a linear system under sufficiently responsive and adaptive conditions, the individual estimation of signal shapes based on RNNs or R-CNNs would lead to accurate weighted superposition and the estimate of the observed induced current/voltage signal. Hence, knowing the individual impulse responses, and their adaptive estimation, a sequence detector can be employed to estimate the base sequences.

In a third claim, in an alternative post-processing method, it is appreciated that as the nucleotides pass through the nanopores, there will be multiple and dependent signals measured. A conventional RNN would not work in this case as it expects a one-dimensional time series. Therefore, multiple independent RNNs can be employed that can be run without using the inherent dependency between the measured signals and plus the coupling. RNN outputs are finally combined through simple majority voting to have the final decision on the sequence of nucleotides.

In a fourth claim, in alternative methodology, assuming three nanopores as shown in FIG. 1, the raw base signals can be post-processed in the following way: First, the top signal I_{T}(t) is shifted by Δ₁ to the right, then the bottom signal I_{B} (t) is shifted by Δ₂ to the left. These signals go through signal padding to have the same length or pad if need be in the streaming mode. Next, these signals are sampled with appropriate periods to get the signal samples. Finally, a recurrent CNN (R-CNN) [1] (f_{R-CNN}(.,.,.)) is implemented to use these signal samples all at the same time and exploit the dependencies/correlations and/or eliminate coupling inherent to their generation. In other words, the R-CNN output consists of samples of the function

f_{R-CNN}(I_{T}(t−Δ₁),I_{M}(t),I_{B}(t+Δ₂)) (Equation 2)

This technique still uses an end-to-end neural network and could be quite complex to implement, particularly in the context of a 100 million stacked nanopore architecture.

In a fifth claim, in another embodiment, neural networks are used to estimate signal shapes for each nanopore rather than doing a joint base calling. The estimation of signal shapes might be different for each physical nanopore. However, with coupling between such nanopores, techniques like R-CNN could be used to estimate signal shapes jointly. For instance in an embodiment of a three nanopore structure, there can be 12 different signal shape estimates, one for each nanopore and base. Next, using such signal estimates, a maximum likelihood detector (MLD) can be employed based on a trellis structure (for each nanopore individually) whose branch metric computations will be done based on the signal estimates that are jointly generated. The basecalling output would be the least costly path in the trellis given the nanopore signal output. Finally, a majority vote at the end merges these sequences to make a decision on a single base sequence. In this case, multiple MLDs per nanopore would be needed. To give an example, consider the following sequence as shown in Table 1:

TABLE 1
Initial Sequencing Detected

| | t = 0 | = t1 | t = 2 | t = 3 | t = 4 | t = 5 | t = 6 | t = 7 | t = 8 | t = 9 | t = 10 | t = 11 | t = 12 | t = 13 | t = 14 |
|---|---|---|---|---|---|---|---|---|---|---|---|---|---|---|---|
| Pore 1 | A | C | T | G | A | C | G | G | C | T | G | A | C | C | A |
| Pore 2 | o | A | C | T | G | A | C | G | C | C | T | G | A | C | C |
| Pore 3 | o | o | A | C | T | G | A | C | G | C | C | T | G | A | C |

Now, assume that even if joint cost estimation, etc. is used, there is a base deleted during the detection process due to faster translocation than usual. So, the following picture can be obtained after a deletion in one of the pores, as shown in Table 2.

Deletion in Pore 3

TABLE 2
Sequencing Detected After A Deletion in Pore 3

| | t = 0 | = t1 | t = 2 | t = 3 | t = 4 | t = 5 | t = 6 | t = 7 | t = 8 | t = 9 | t = 10 | t = 11 | t = 12 | t = 13 | t = 14 |
|---|---|---|---|---|---|---|---|---|---|---|---|---|---|---|---|
| Pore 1 | A | C | T | G | A | C | G | C | C | T | G | A | C | C | A |
| Pore 2 | o | A | C | T | G | A | C | G | C | C | T | G | A | C | C |
| Pore 3 | o | o | A | C | T | G | A | C | G | C | T | G | A | C | C |

As shown in Table 2, a deletion in pore 3 happens right after t=8, where a nucleotide C is deleted by the pore due to translocation or detection problems. By considering the output of all three pores, this deletion error can easily be detected and corrected through some majority logic voting system.

In a sixth claim, as an alternative to the fifth claim, the MLD detectors (for each nanopore) can exchange information during the sequence estimation process to decide on the single base sequence while sequencing their own bases. In other words, while calculating the distance metrics, corresponding distance metrics from other trellises can be used to determine the most likely sequence. Thus, in this formulation, bases are jointly determined and MLDs work collaboratively. That is to say, MLDs converge to the same sequence decision while moving over their corresponding signal sequences. The joint collaboration results in the same consensus over the most likely base sequence by identifying errors, deletions as well as insertions to the base sequence. A short-time memory would need to be used for back-tracing in the MLD implementation. However, due to time dependence between sequences, memory used for each MLD can help other memories in the back-tracing process.

In a seventh claim, in another embodiment of the system for the example number of nanopores of FIG. 1 and apparatus described therein, the contributions of distance metrics of the corresponding MLDs can be weighted in a unique way. The main reason behind it is due to the preprocessing of the fourth claim noted above, where the top and bottom signals are shifted to the right and left by different amounts in a 3-nanopore joint base calling and natural translocation speeds of nanopores are different by design. However, these estimations are subject to errors and/or failures which can be detrimental to the overall system detection performance. Particularly, if these parameters become non-adaptive due to the varying translocation speeds and environmental changes (such as PH for biological nanopores), these shift amounts may not be accurate throughout the sequencing process. In the case of adaptive calculation, a highly non-stationary signal nature can make these parameter estimations hard to be of use in practice. In an embodiment of the idea, the middle nanopore may be manufactured to give the best performance while the other neighboring nanopores can be structured as helpers and can be chosen to be cost-efficient and of lesser quality to reduce overall cost. For instance, the middle nanopore can be larger in size, can use the best and more costly chemical processes, can use extra mechanisms to stabilize the translocation, etc. Thus, the MLD for the middle nanopore current output forms the main detection engine while the other two MLDs can act as auxiliary detection engines and their metric information can be weighted less as compared to the main engine. In this manner, errors in the shift amount estimation would be less propagated to the main sequence estimation process to ensure better detection performance. In fact, the shift amounts Δ₁ and Δ₂ and the weights are interconnected to each other and need to be optimized jointly.

In an eighth claim, in still another embodiment of the storage system, data could be encoded using indel-correction code, followed by a product code able to correct both substitution errors and erasures. This concatenation of coding could be necessary to reduce error rates below 10⁻²⁰ nucleotide detection error rates. Through joint detection, some of the indels would be detected due to the diversity of multiple captured copies of the same data. These detected nucleotides are filled/labelled as erasures to be used by the subsequent product decoding. Product codes are great selections to attack a mixture of substitution errors and erasures whereas the front-end indel-correcting code will take care of the remaining single deletions or insertions. The remaining indels are expected to be small in size, such as a single indel per codeword at maximum.

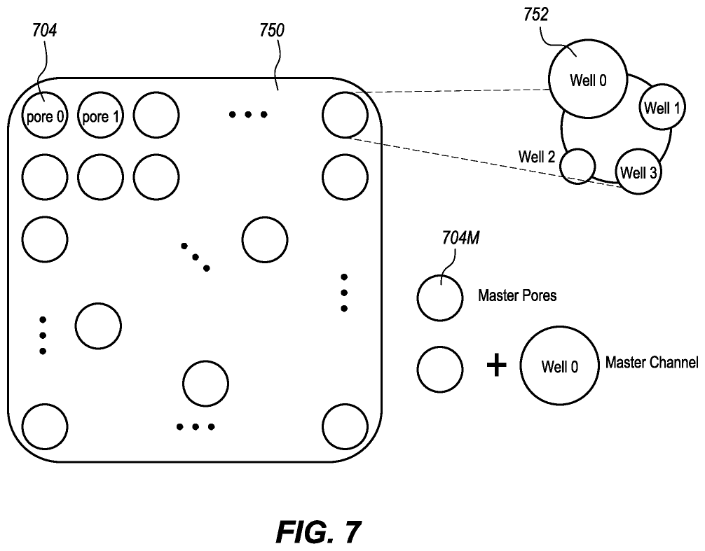

In a ninth claim, in yet another embodiment of the proposed storage system, the concept of “Master channel” can be used to periodically learn the signal shapes, filter coefficients, whitener coefficients, branch metrics, shift amounts, pad amounts, and sampling periods among other parameters of the storage system. Master nanopores have a special chemical header attached to the nanopore entrance. This chemical composition identifies specially designed DNA reference molecules. These nanopores do not allow any other molecule to pass but these special molecules. Therefore, since these reference molecules are known, corresponding system parameters are optimized based on the resulting nanopores. These parameters are then communicated with non-master nanopores for update during real-time sequencing operation. FIG. 7 is a simplified schematic cross-sectional view illustration of nanopores 704 usable within the nucleic acid digital data storage system 100 illustrated in FIG. 1 shown on a two-dimensional planar surface 750. As shown, each stacked nanopore 704 is associated with multiple wells 752. In this example, four wells 752 are shown for each nanopore 704 just like in an Oxford Minion Device.

As can be seen in FIG. 7, well-sizes are different and a nanopore 704 can only switch to one and only one of these wells 752 (forming the DNA channel) during sequencing. Well sizes are different because DNA molecules pass more frequently with the bigger size wells 752. Hence, by switching between the wells 752 for master nanopores 704M, the update frequency of the system parameters can be adjusted. The switch between different wells 752 in other non-master channels is done based on the probabilities of DNA molecules passing through each well 752. For example, the biggest well 752 can be switched on for 50% of time, whereas the rest of the wells 752 share equally the other 50% of the time. The number of and the allocation of the master nanopores 704M among all the set of nanopores 704 are adjusted such that enough update information can be collected and allocation is balanced all across the two-dimensional surface 750 such that the separation between the master nanopores 704M is maximized for a given fixed number.

It is further noted that thanks to their solid-state nature, the nanopores 704 are expected to survive in their initial state for a long time and hence ensure a stationary signal shape throughout the data lifetime. In case a major change is detected in the storage system 100, retraining of collected data is executed to correct the signal shapes and sampling times. Otherwise, a drift in the storage system 100 may dramatically reduce the detection accuracy performance of the subsequent detection algorithms.

It is further appreciated that other machine learning schemes can also be used within the context of this disclosure where appropriate as long as multi-class classification is performed. For instance, the regression or reinforcement learning can be used to estimate h₁(t) and h₂(t). Depending on the nanopore model, signal levels can be mapped to these functions provided the sampling periods are known. Another such example is Error Correction Output Coding (ECOC) frameworks, in which multiple component binary classifiers are used with an appropriate merging algorithm to achieve successful multi-class classification. All multi-class (4-class) classification algorithms can be used to classify bytes in each iteration into one of the four classes A, G, C, T. Accuracy of such algorithms is of crucial importance for the iterations to work properly and in order not to introduce new type of errors into the decoding operation. Depending on the technique, the training may take different amounts of time and memory space.

With the present invention, contrary to the state-of-the-art, Recursive Neural Networks (RNNs) are used to estimate the signal shapes for each base nucleotide rather than using a base detection process directly. Based on the estimated signal shapes, the data storage system is configured to use Recurrent Convolutional Neural Networks (R-CNNs) and conventional detection algorithms based on estimated signal shapes such as noise predictive maximum likelihood detection (NPMLD) to sequence the nucleotides in a spatially coordinated way. In this manner, improved detection accuracy performance is ensured, while giving a brand-new methodology to the detection process within the context of explainable AI and low-complexity information decoding.

More specifically, first, the data storage system is configured to use multiple pores put on top of each other where their sizes, architecture of their internal structure, and what they are made of, may be different. In fact, hybrid pores (both protein and solid-state at the same time) could be combined to make up the multi-pore architecture. Protein nanopores are robust, easily reproducible at low cost, and easy to modify. On the other hand, solid-state nanopores, due to their chemical nature, would improve the cost and scale of nanopore analyses. So, within this architecture, the present invention can use the best of both worlds to improve the detection process. It is appreciated that for compatibility to solid-state circuit development, allowing solid-state-only nanopores may be preferable from a manufacturing cost point of view.

Another objective of such a design is to create almost-balanced translocation speeds so as to ensure stationary system and signal shapes over a long period of time. Thus, another novelty of the present invention is the ability to control the translocation time of DNA molecules through the use of multiple pores which may be interleaved with different sized cavities. Through the use of multiple pores and using multiple chemical mechanisms to generate a driving force inside the cavities, an almost constant translocation time is aimed. In fact, pores would help each other to rearrange the speed if it becomes too fast or too slow. The system can be further configured to detect signal anomalies and have to trigger re-estimation of signals (offline) to maintain detection performance (for the later detection processes). Fastest translocation is expected at the top of the pores, whereas the slowest translocation speeds are associated with the bottom of the stacked pore structure.

In summary, the present disclosure describes a methodology based on multi-pore sequencing to improve the base-calling performance through redundancy in space, thereby adding a spatial resolution into the detection process. The classic approach to improve spatial resolution is to decrease k (ideally to 1, thus using all single-base detection studies through miniaturizing the pore sizes). However, with the present invention, the k value is artificially increased through stacking multiple nanopores inside a membrane, with each housing one or more nucleotides at a given time. Moreover, the present invention is configured to use noise predictive data detection algorithms and error/erasure/deletion and insertion correction codes to introduce redundancy in time and reduce the complexity. By introducing these two redundancies at the same time, and by decoupling the system components, the data storage system aims to improve the detection speed and accuracy performances of the nanopore sequencing process.

Thus, with use of the data storage system configured having features and aspects of the present invention, certain disadvantages can be overcome. For example, the present invention can be utilized to overcome at least these three important problems with respect to the state of the art: (1) Neural network-based detection approach requires complex and/or specially designed hardware. Moreover, hundreds of such would be needed to do parallel processing; (2) It is impossible to reason about the overall base-detection process and hence hard to improve the system accuracy performance through introducing novel system modules/algorithms. In fact, in all conventional systems, all signal time-dependent disturbances such as noise, inter-symbol interference, phase shift, signal smearing, etc., are solved by RNNs in a complicated way; and (3) Nanopore sequencing is based on ionic current blockade levels and single-dimensional temporal data. In other words, there is no spatial data component to enhance detection performance and hence this results in high error rates.

It is understood that although a number of different embodiments of the data storage system have been illustrated and described herein, one or more features of any one embodiment can be combined with one or more features of one or more of the other embodiments, provided that such combination satisfies the intent of the present invention.

While a number of exemplary aspects and embodiments of the data storage system have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

What is claimed is:

1. A nucleic acid digital data storage system that uses nanopore sequencing to read data values chemically embedded in oligonucleotides, the nucleic acid storage system comprising:
a membrane having a plurality of nanopores that are stacked upon one another in a multi-nanopore arrangement;
a voltage source that is configured to direct voltage across the plurality of nanopores; and
a nucleic acid strand including the oligonucleotides that is threaded through each of the plurality of nanopores within the membrane.

2. The nucleic acid digital data storage system of claim 1 wherein the nanopores are surrounded by an electrolyte solution within the membrane.

3. The nucleic acid digital data storage system of claim 1 wherein the nucleic acid strand is a DNA strand; and wherein the oligonucleotides include one or more of adenine, guanine, cytosine, and thymine.

4. The nucleic acid digital data storage system of claim 1 wherein the nucleic acid strand is an RNA strand.

5. The nucleic acid digital data storage system of claim 1 wherein the voltage from the voltage source is applied across each of the plurality of nanopores independently of one another to create an electrical field across pore ends of each of the plurality of nanopores; and wherein the electrical field creates an ionic current to pass through each of the plurality of nanopores.

6. The nucleic acid digital data storage system of claim 1 wherein the membrane is usable to capture multiple waveforms for a base sequence when the oligonucleotides are threaded through the plurality of nanopores; and wherein the oligonucleotides being threaded through each of the plurality of nanopores generates a corresponding ionic current.

7. The nucleic acid digital data storage system of claim 6 wherein a separate base signal is generated from the nucleic acid strand being threaded through each of the plurality of nanopores.

8. The nucleic acid digital data storage system of claim 7 wherein Recursive Neural Networks are used to estimate a signal shape for each oligonucleotide.

9. The nucleic acid digital data storage system of claim 8 wherein Recurrent Convolutional Neural Networks and noise predictive maximum likelihood data detection algorithms are used based on the estimated signal shapes to sequence the oligonucleotides.

10. The nucleic acid digital data storage system of claim 7 wherein each of the base signals is modified by each of a post-processing system, a joint symbol detection system, and an Error Correction Coding (ECC) decoding system.

11. The nucleic acid digital data storage system of claim 1 wherein the plurality of nanopores includes a first nanopore, a second nanopore and a third nanopore that are stacked one on top of another from top to bottom in the multi-nanopore arrangement; and wherein the membrane further includes a first cavity that is defined between the first nanopore and the second nanopore, and a second cavity that is defined between the second nanopore and the third nanopore.

12. The nucleic acid digital data storage system of claim 11 wherein each of the plurality of nanopores is different from each of the other nanopores in one or more of size and translocation speed.

13. The nucleic acid digital data storage system of claim 12 wherein the first cavity has a first size, and the second cavity has a second size that is different than the first size.

14. The nucleic acid digital data storage system of claim 1 wherein the membrane is one of a biological membrane, a solid-state membrane, and a hybrid of a biological membrane and a solid-state membrane.

15. A method for using nanopore sequencing to read data values chemically embedded in oligonucleotides, the method comprising the steps of:
stacking a plurality of nanopores upon one another in a multi-nanopore arrangement within a membrane;
directing voltage across the plurality of nanopores with a voltage source; and
threading a nucleic acid strand including the oligonucleotides through each of the plurality of nanopores within the membrane.

16. The method of claim 15 further comprising the step of providing an electrolyte solution within the membrane so that the nanopores are surrounded by the electrolyte solution.

17. The method of claim 15 wherein the step of directing includes applying the voltage from the voltage source across each of the plurality of nanopores independently of one another to create an electrical field across pore ends of each of the plurality of nanopores; and creating an ionic current with the electrical field to pass through each of the plurality of nanopores.

18. The method of claim 15 further comprising the steps of capturing multiple waveforms for a base sequence with the membrane when the oligonucleotides are threaded through the plurality of nanopores; and generating a corresponding ionic current from the oligonucleotides being threaded through each of the plurality of nanopores.

19. The method of claim 18 further comprising the steps of generating a separate base signal from the nucleic acid strand being threaded through each of the plurality of nanopores; estimating a signal shape for each oligonucleotide using Recursive Neural Networks; and sequencing the oligonucleotides using Recurrent Convolutional Neural Networks and noise predictive maximum likelihood data detection algorithms based on the estimated signal shapes.

20. The method of claim 19 further comprising the step of modifying each of the base signals by each of a post-processing system, a joint symbol detection system, and an Error Correction Coding (ECC) decoding system.
